(19) United States
(12) Patent Application Publication
(10) Pub. No.: US 2009/0114218 A1
(43) Pub. Date: May 7, 2009

(54) ELECTROTHERAPEUTIC TREATMENT DEVICE AND METHOD

(75) Inventor: Bradley Delton Veatch, Westminster, CO (US)

Correspondence Address: SHERIDAN ROSS PC, 1560 BROADWAY, SUITE 1200, DENVER, CO, 80202, US

(73) Assignee: ADA TECHNOLOGIES, INC., Littleton, CO (US)

(21) Appl. No.: 11/734,626

(22) Filed: Apr. 12, 2007

Related U.S. Application Data
(60) Provisional application No. 60/792,221, filed on Apr. 13, 2006.
(60) Provisional application No. 60/802,271, filed on May 19, 2006.

(51) Int. Cl. A61M 15/02; A61M 37/00

(52) U.S. Cl. 128/202.25; 604/25

(57) ABSTRACT

The present invention is directed to a treatment method and system that (a) while controlling ozone production, electrically charges a plurality of (i) atomic particles (e.g., diatomic oxygen and water molecules) and/or (ii) electrically charged droplets in an input gas stream to form a charged gas stream and (b) provides the charged gas stream to a living organism to be treated.

### CROSS REFERENCE TO RELATED APPLICATION

The present application claims the benefits of U.S. Provisional Application Ser. No. 60/792,221, filed Apr. 13, 2006, and 60/802,271, filed May 19, 2006, each of which is entitled “Electrotherapeutics Using Ionization and Electrospray as Energy Transfer Mechanisms” and incorporated herein by this reference.

### STATEMENT REGARDING FEDERALLY SPONSORED RESEARCH OR DEVELOPMENT

The U.S. Government has a paid-up license in this invention and the right in limited circumstances to require the patent owner to license others on reasonable terms as provided for by the terms of Contract No. 1R43EY016609 awarded by the National Eye Institute.

### FIELD OF THE INVENTION

The invention relates generally to electrotherapeutics and particularly to the generation and use of electrical charge and/or electrically charged particles to treat and prevent disorders in living organisms.

### BACKGROUND OF THE INVENTION

Electrotherapeutics generally encompass the application of electrical phenomena to medical applications. Examples of electrotherapeutics include, for example, pacemakers, electrocardiograms, defibrillators, to name but a few applications. In one application, electrodes are physically contacted with tissue to foster wound healing. These methods generally require intimate electrical contact between conductive terminals and the subject tissue. Direct physical contact with the patient is not always possible, such as for burn patients, and, even if possible, can be discomforting to the patient.

Some experimental electrotherapeutic work has been performed in which airborne, charged particles were inhaled by patients. For example, Wehner in “Technical and Clinical Aspects of Electro-Aerosols in Inhalation Therapy”, San Diego Sym Bio Engineering, pp. 32-38 (1963), documents the use of electrically charged aerosol droplets on patients with generally beneficial effects. Krueger, et al., in “Electric Fields, Small Air Ions, and Biological Effects”, Int J Biometeor 1978, vol. 22, Num. 3, pp. 202-212, documents increases in beat frequency of ciliated tracheal tissue using ions generated through radioactive soft beta decay in air. Palti, et al., in “The Effect of Atmospheric Ions on the Respiratory System of Infants”, Pediatrics, vol. 38, no. 3, September 1966, pp. 405-411, documents the exposure of infants with asthma to negatively and positively charged ions to observe qualitative changes in asthma attack severity against a control group. The papers establish that both electrically charged droplets and air ions appear to cause a beneficial response in patients exposed to them. While beneficial effects were observed for negative ions in these papers, specific numbers of charged species or ions absorbed (dose) were not measured nor was the dosage of charged particles controlled.

As electrotherapeutics have increased in popularity and efficacy, ionizing air cleaners have also grown in popularity for home and office use. Also known as electrostatic precipitators, ionizing air cleaners trap charged particles on oppositely electrically charged plates. This is done by electrically charging airborne particles with a first electrical polarity and trapping the charged particles on collection plates having an opposite second electrical polarity. For example, the first electrical polarity can be negative, and the second positive. In the process of charging particulates in the air, charged oxygen molecules, namely charged diatomic and triatomic oxygen molecules, are formed. Triatomic oxygen molecules are also known as ozone. Unlike ozone in the upper atmosphere, which helps shield us from harmful ultraviolet rays, ozone near ground level is an irritant that can aggravate asthma and decrease lung function. Experts agree that an ozone concentration of more than 80 ppb for eight hours or longer can cause coughing, wheezing, and chest pain while worsening asthma and deadening the sense of smell in those who inhale it. It also raises sensitivity to pollen, mold, and other respiratory allergy triggers and may cause permanent lung damage. Longer term exposure to ozone has been linked to higher, premature death rates. Ozone can create other pollutants; for example, ozone reacts with the terpenes in lemon- and pine-scented cleaning products and air fresheners, creating formaldehyde, a known carcinogen, and other irritants, including ultrafine particles that can go deep into the lungs. At least one consumer reporting agency has tested ionizing air cleaners and concluded that they expose users to significant amounts of ozone. Stated another way, while ionizers can be effective in removing particulates, such as dust, smoke, and pollen, from the air and address one set of health problems, the “cleaned” air can have potentially harmful amounts of ozone, thereby creating a host of other health problems for users.

### SUMMARY OF THE INVENTION

These and other needs are addressed by the various embodiments and configurations of the present invention. The invention is directed generally to electrotherapeutics using charged particles to transfer electric charge and/or electrical energy to an organism to be treated.

In one embodiment of the present invention, a treatment method is provided that includes the steps:

(a) while controlling ozone production, electrically charging a plurality of (i) atomic particles (e.g., diatomic oxygen and water molecules) and/or (ii) electrically charged droplets in an input gas stream to form a charged gas stream; and

(b) providing the charged gas stream to a living organism to be treated.

While not wishing to be bound by any theory, charged particles, such as diatomic molecular oxygen, are believed to transfer electrical energy, or free electrons, to the living organism and can have a number of beneficial effects. The effects include inducing beneficial physiological responses in the treated tissue. Examples of beneficial responses include secretion of mucin granules from goblet cells, such as on the respiratory tract and sinus linings and the ocular surfaces, stimulation of endocrine glands, such as the meibomian and lacrimal glands of the eye, to produce essential tear film components, modification of electrical charge distributions of mucosa and mucus secretions in the respiratory tract (e.g., lungs, airways, sinuses, and nasal passages) that improve the elimination of mucus from those tracts or modify the physical properties of the mucus making its elimination from those tracts easier for the body's structures to accomplish, the release of bioactive chemicals, the destruction of microbes, such as parasites and pathogens (e.g., bacteria, fungi, and viruses) attacking topically the treated tissue to prevent or lessen infection or detrimental effects, and accelerated rates of wound healing. Exemplary physical conditions that may be treated by the present invention include respiratory ailments (e.g., asthma, cystic fibrosis, and sinus disorders), ocular conditions (e.g., dry eye syndrome, aqueous deficient dry eye, evaporative dry eye, keratoconjunctivitis sicca, ocular allergies such as vernal conjunctivitis, Sjogren's Syndrome, and evaporative disorders), allergies, thermal and chemical burns, and post-surgical wounds.

The method can further control the charged particle dosage amount and/or rate to substantially optimize treatment efficacy. Typically, the dosage is controlled by changing the voltage difference between the charging device and the living organism being treated and/or by regulating the flow velocity of the charged gas provided to the organism.

The method can further provide for automated selection of the appropriate treatment protocol and self-configuration of the electrotherapeutic treatment system. The treatment protocol is typically selected by an operator, and an identifier of the selected treatment protocol mapped against a lookup table to determine the parameter settings to be employed and the instructions to be provided to the operator to implement the protocol.

The present invention can provide a number of advantages depending on the particular configuration. By way of example, the invention can effect therapeutic treatment of tissue without direct physical, or intimate, contact of the charging device with the tissue being treated. This can be important for certain types of conditions being medicated, such as burns and optical disorders where patient sensitivity and the danger of infections are concerns. It can control production of ozone, which can be harmful, or even fatal over the long term, to patients. It can produce beneficial results, including increased cellular secretion, increased rates of cell migration, increased rates of cell proliferation, and reduced rates of evaporation of the ocular tear film. It can effect efficacious treatment of physiological conditions without the need to introduce pharmaceutical agents to the patient's gastrointestinal tract, thereby diminishing the risk of adverse allergic reactions. It can be used by any patient regardless of the patient's physical condition. It has no known risk of allergic or undesirable side reactions, particularly when the charge polarity is negative. No abnormal adverse cellular processes or oncogenic effects have been observed in experiments performed to date. It can be nonintrusive and initiated and terminated at the will of the patient, thereby encouraging higher levels of patient use and treatment protocol compliance. It can be embodied not only as a clinical treatment system but also as a system that ordinary consumers can use at home, in a vehicle, and at work. It can be embodied as an inexpensive system that is readily affordable not only by health care providers but also by consumers.

These and other advantages will be apparent from the disclosure of the invention(s) contained herein.

As used herein, “at least one”, “one or more”, and “and/or” are open-ended expressions that are both conjunctive and disjunctive in operation. For example, each of the expressions “at least one of A, B and C”, “at least one of A, B, or C”, “one or more of A, B, and C”, “one or more of A, B, or C” and “A, B, and/or C” means A alone, B alone, C alone, A and B together, A and C together, B and C together, or A, B and C together.

It is to be noted that the term “a” or “an” entity refers to one or more of that entity. As such, the terms “a” (or “an”), “one or more” and “at least one” can be used interchangeably herein. It is also to be noted that the terms “comprising”, “including”, and “having” can be used interchangeably.

The term “automatic” and variations thereof, as used herein, refers to any process or operation done without material human input when the process or operation is performed. However, a process or operation can be automatic even if performance of the process or operation uses human input, whether material or immaterial, received before performance of the process or operation. Human input is deemed to be material if such input influences how the process or operation will be performed. Human input that consents to the performance of the process or operation is not deemed to be “material”.

The terms “determine”, “calculate” and “compute,” and variations thereof, as used herein, are used interchangeably and include any type of methodology, process, mathematical operation or technique.

The term “module” as used herein refers to any known Or later developed hardware, software, firmware, artificial intelligence, fuzzy logic, or combination of hardware and software that is capable of performing the functionality associated with that element. Also, while the invention is described in terms of exemplary embodiments, it should be appreciated that individual aspects of the invention can be separately claimed.

The above-described embodiments and configurations are neither complete nor exhaustive. As will be appreciated, other embodiments of the invention are possible utilizing, alone or in combination, one or more of the features set forth above or described in detail below.

### BRIEF DESCRIPTION OF THE DRAWINGS

FIG. 1 is a block diagram depicting an electrotherapeutic treatment system according to a first embodiment of the invention;

FIG. 2 is a block diagram depicting in greater detail elements of the electrotherapeutic treatment system of FIG. 1;

FIG. 3 is a cross-sectional view of an electrotherapeutic treatment system according to a second embodiment of the present invention;

FIG. 4 is a cross-sectional view of an electrotherapeutic treatment system according to a third embodiment of the present invention;

FIG. 5 is a cross-sectional view of an electrotherapeutic treatment system according to a fourth embodiment of the present invention;

FIG. 6 is a cross-sectional view of an electrotherapeutic treatment system according to a fifth embodiment of the present invention;

FIG. 7 is a block diagram depicting an electrotherapeutic treatment system according to a sixth embodiment of the present invention;

FIG. 8 depicts target containment according to a seventh embodiment of the present invention;

FIG. 9 depicts a set of lungs treated by the system of FIG. 8;

FIG. 10 depicts an ear canal applicator according to an eighth embodiment of the present invention;

FIG. 11 depicts an electrotherapeutic treatment system according to a ninth embodiment of the present invention;

FIG. 12 is a flow chart depicting a treatment protocol selection application according to a tenth embodiment of the present invention;

FIG. 13 depicts a set of data structures useful with the treatment protocol selection application of FIG. 12;

FIG. 14 is a perspective view depicting an experimental apparatus used in the set of experiments;

FIG. 15 is a bar chart of glycoprotein secretion (fold increase) (vertical axis) against control and ionization tests (horizontal axis);

FIG. 16 depicts control and ionization sample wells from a set of experiments;

FIG. 17 is a bar chart (fold increase) (vertical axis) against control and ionization tests (horizontal axis);

FIG. 18A is a plot of relative amount of the DNA (vertical axis) against exposure time (minutes) (horizontal axis);

FIG. 18B is a plot of DNA synthesized (vertical axis) against exposure time (minutes) (horizontal axis);

FIG. 19A is a plot of cell count (vertical axis) against exposure time (minutes) (horizontal axis); and

FIG. 19B is a plot of cell count (vertical axis) against exposure time (minutes) (horizontal axis).

### DETAILED DESCRIPTION

### Overview of Electrotherapeutic Treatment System

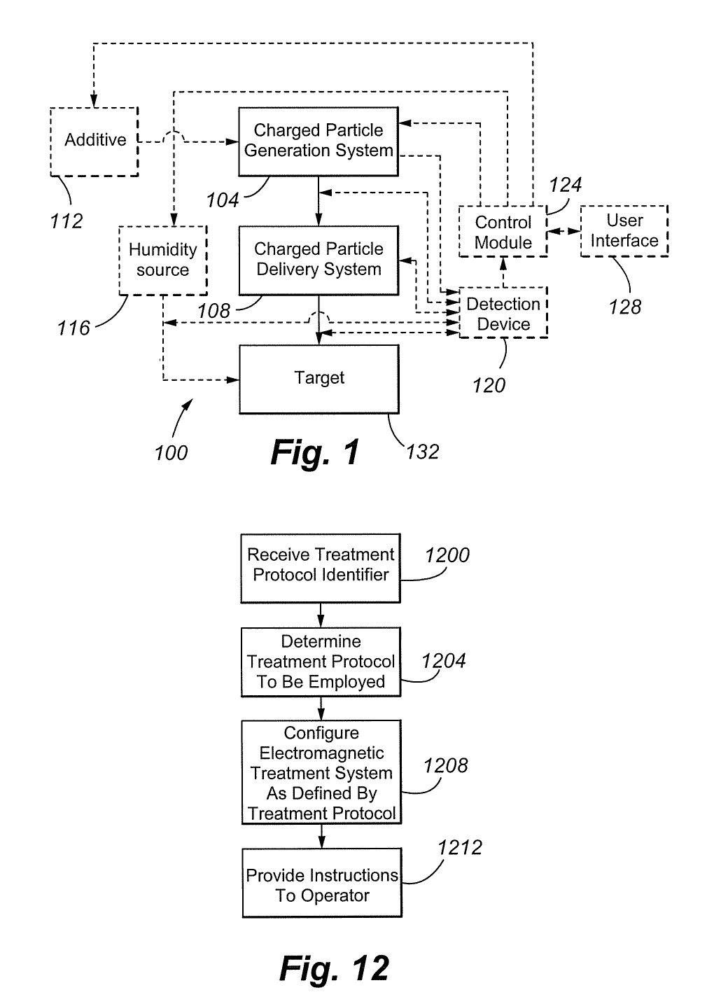

FIG. 1 depicts an electrotherapeutic treatment system 100 according to a first embodiment of the present invention. The system 100 includes a charged particle generation system 104, charged particle delivery system 108, an additive (optional) 112, a humidity source (optional) 116, a detection device (optional) 120, a control module (optional) 124, and a user interface (optional) 128. The system 100 provides a gas stream containing charged particles, preferably at the atomic, molecular, and/or macromolecular (e.g., aerosol) scales, to a target 132. Typically, the maximum size of the charged particle is less than 3 microns.

The system 100 applies controlled quantities of electric charge, of a selected polarity and using an airborne transport mechanism, to target specific cells or tissues of the target organism. Charged particles can include ionized molecules and/or charged droplets, typically of more complex molecules. For most applications, negative charge is preferred; however, this does not eliminate positive or proprietary charge ratios, or time-dependent variations of these ratios that may prove beneficial.

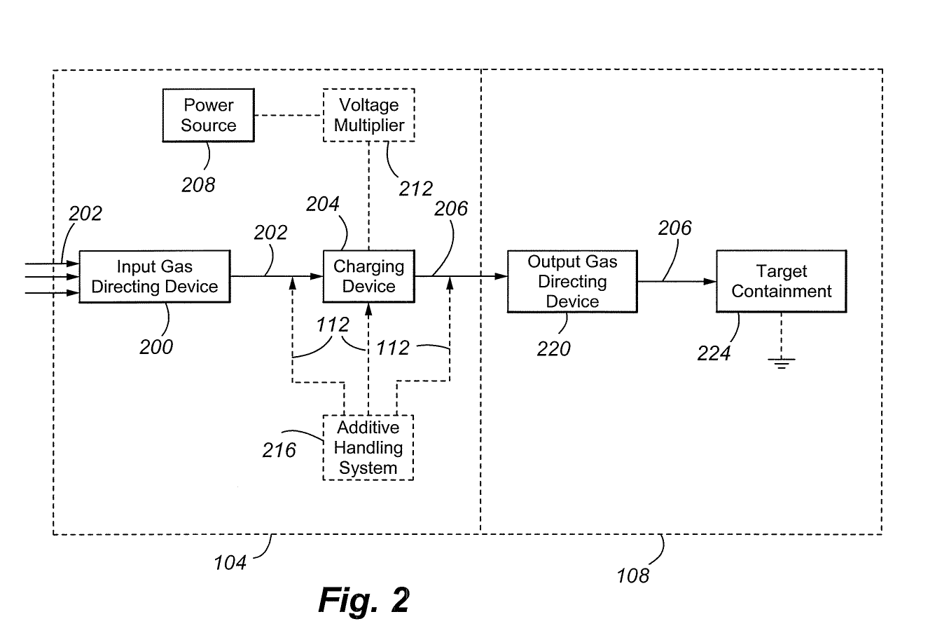

An embodiment of the charged particle generation and delivery systems 104 and 108 is depicted in FIG. 2. The charged particle generation system 104 includes an input gas directing device 200, charging device 204, power source 208, voltage multiplier (optional) 212, and additive handling system (optional) 216. The charged particle delivery system 108 includes an output gas directing device 220 and target containment 224.

The input gas directing device 200 is any assembly for directing an input gas 202 to the charging device 204. The input gas directing device 200 typically includes a fan or positive or negative pressure pump for pressurizing the input gas 202 or a pressurized stored gas source. The pressure propels the gas towards the charging device 204 and a suitable housing to provide containment.

The charging device 204 can be any suitable component(s) for applying a positive or negative electrical charge to particles in the gas stream 202. In one configuration, the charging device 204 is a corona discharge needle or electrode or set of corona discharge needles or electrodes positioned in the gas stream 202 to produce an electric wind. In another configuration, ions are produced using a radioactive source, such as tritium, to produce ions in the gas stream 202 through localized gas molecule ionization and an electrostatic screening device (not shown) that permits ions of the desired polarity to pass through for transport to the target 132.

While not wishing to be bound by any theory, when the gas is air it is believed that the charging device provides an additional electron to a substantial portion of, and preferably at least most of, the diatomic oxygen molecules to form superoxides, i.e., O₂−, in the charged input gas stream. Small molecules containing oxygen, such as diatomic oxygen and water, are the preferred carrier for the negative charge. However, certain molecules, such as carbon monoxide and carbon dioxide, have been found to be undesirable due to a potentially deleterious effect on pulmonary function. It is not known whether it is the electrical energy proper and/or the charged species involved that induces biological effects, and the mechanisms may play differing roles for differing medical conditions. In another configuration, the charging device 204 is an electrically charged conduit, preferably a nebulizer tip, and uses a solution-based ionization mechanism to from an electrospray, including electrically charged droplets of an additive 112. As in the case of the prior configurations, a substantial portion, and preferably at least most, of the liquid particles are charged. Any other source for electrically charged ions can be used as the charging device, including particle accelerators, free electrons produced through photovoltaic effects, and the like.

The power source 208 and optional voltage multiplier 212 are selected to provide no more than a selected amount of ozone formation in the charged gas stream. In most applications, the voltage applied to the charging device 204 is preferably maintained at a level to reduce the amount of ozone produced to no more than about 80 ppb in the charged gas and, even more preferably, to no more than about 60 ppb. In one configuration, this is accomplished by maintaining the preferred voltage at the charging device 204 (e.g., corona discharge needle or nebulizing tip) to no more than about −20 kvolts, more preferably to no more than about −15 kvolts, and, even more preferably, from about −7 to about −13 kvolts. Although the power source 208 can be Alternating Current (“AC”) or Direct Current (“DC”), AC is preferred, with the AC being rectified (not shown) to DC prior to or within the multiplier 212 and charging device 204. The voltage multiplier 212 is preferably a Cocroft-Walton multiplier ladder (using fractional picofarad doubling capacitors), an influence machine, frictional or triboelectric machine, or an inductive static machine. Examples of inductive static machines include Wimshurst and Toepler machines. A preferred voltage multiplier 212 is a Cocroft-Walton multiplier ladder having adjustable output between about −7 KVDC and −16 KVDC for dose control.

Preferably, the voltage output by the power source 208 and/or voltage multiplier 212 is maintained at a substantially constant magnitude. More preferably, the output or applied voltage varies no more than about 5%, and even more preferably no more than about 2%, over substantially the entire time that the charging device 204 is in operation.

The additive handling system 216 provides the additive 112 directly to the charging device 204 or upstream or downstream of the device. The additive 112 can be any liquid solution, whether acting simply as a charge carrier having no inherent medicinal properties or a liquid pharmaceutical agent or both. Preferred charge carriers include salinated water and desalinated water. Pharmaceutical agents can be electrically uncharged (or neutral) or be positively or negatively charged. In one configuration, the charging device charges the droplets with a charge opposite to the natural charge of the additive 112 to neutralize substantially, or even reverse, the natural charge of the droplet. Examples of pharmaceutical agents include antimicrobial agents, bronchodilators of the Beta 2 agonist and anticholinergic types, inhalants for reducing or altering mucus viscosity, and mixtures thereof. The pharmaceutical agents can be in the form of a finely sized solid particle entrained in a liquid carrier or different liquid pharmaceutical agent or in the form of a solution in which the agent is dissolved in a liquid carrier or different liquid pharmaceutical agent. The additive handling system 216 typically includes one or more pumps and a conduit to transport the additive 112 from a receptacle to the charging device 204. As will be appreciated, a gravity feed of the additive may also be used.

The output gas directing device 220 is any assembly for directing a charged output gas 206 to the target 132 or target containment 224. Transport may or may not be augmented using a fan, shrouds, tubing, directed gas jet, or electric and magnetic fields to deliver ions or charged droplets to the target 132. The output gas directing device 220 typically includes a fan or positive or negative pressure pump for pressurizing the output gas. In one configuration, the pressure of a stored pressurized gas source is sufficient to move the input gas 202 through the system 100 and direct the charged output gas 206 to the target or target containment. The directing device 220 can include suitable a conduit to transport the charged, output gas 206 to or direct the gas towards the target 132. In another configuration, charged particles, or ions, are emitted from a corona discharge needle and directed at the target 132 in the form of a jet or plume. The shape and velocity of the jet or plume can be controlled using an electromagnetic (e.g., electric or magnetic) field. In another configuration, the charged particles are in the spray emanated by a nebulizer tip or nozzle that is directed towards the target 132.

Target containment 224 can be any suitable enclosure for contacting the charged, output gas 206 with the target 132. Examples of containments include chambers, such as a chamber similar to a hyperbaric chambers in which patient is wholly contained, a more localized enclosure, such as flexible containment, hood, or shrouding, in which only a part or member of the patient is positioned with the remainder of the patient's body being located outside of the containment, or a breathing mask. To control buildup of electrical charge on the surface of the containment 224 and thereby provide a force to repel charged particles away from the target, the containment 224 is preferably grounded. The containment, as well as the conduit transporting the charged, output gas 206 to the containment 224 are preferably selected to inhibit charge dissipation or the formation of electric fields that impede movement of the charged particles and may be grounded. Suitable materials for the containment and conduit include glass and some plastics.

Returning to FIG. 1, the system 100 further includes a humidity source 116 for certain applications. The treatment efficacy of the charged, output gas 206 and patient comfort in certain medical conditions is improved by using a higher-than-ambient humidity in the output gas 206. Examples of such medical conditions include dry eye syndrome, asthma, chronic obstructive pulmonary disease, cystic fibrosis, emphysema, bronchitis, and other respiratory disorders, and burns. Although electrical charge imparted to particles can bleed off more quickly when humidity is present (because water contains an oxygen atom and will assume readily a negative charge), it is believed that the substantial charge imparted to the gas 202 will still have sufficient charge density when contacted with the target 132 to have curative and/or health maintenance effects. Typically, humidity is greater than the humidity level of the input gas 202 (or the external atmosphere) and preferably is at least about 10% greater than the humidity level of the input gas 202. The humidity source can be of any suitable form, including an evaporator, ultrasonic or thermal humidifier, swamp cooler, and the like.

In some applications, it is preferred that the humidity level of the charged, output gas 206 be lower than that of the input gas 202 (or external atmosphere) to inhibit charge bleed off. This humidity reduction can be realized in any suitable manner, such as by contacting the uncharged input gas 202 with a desiccant (packed or fluidized) bed (not shown).

The detection device 120 can be one sensor or a suite of sensors providing signal feedback for system control by the control module 124. Examples of sensors include voltage sensors to monitor the voltage potential applied to or by the charging device 204, electrical current sensors to monitor the electrical current flowing to the charging device 204, gas flow rate sensors to measure the flow rate of the input or output gases 202, 206, or of the additive 112, humidity sensor to determine the humidity level of the input or output gases 202, 206, altimeter to measure altitude above or below sea level, barometer to measure barometric pressure of the external atmosphere, a charge sensor to detect electrical charge in proximity to the target, or area to be medicated (e.g., a known volume of air in a known period of time is drawn into a conducting tube while measuring with a picoammeter (and mathematically integrating) the electric current to ground from the tube, which computation determines the charged particle density), a laser or optical sensor to determine particulate counts in the input or output gas, thermometer to measure the temperature of the input and/or output gases, mass or volume flowrate sensors, and the like.

The control module 124, inter alia, controls system parameters and settings in response to signal feedback from the detection device(s) 120, selects appropriate treatment protocols and configures the system 100 in accordance with the selected treatment protocol, and provides audible and visual feedback to the operator regarding operation of the system 100. As noted, the control module 124 monitors the voltage and current provided to the charging device 204 to maintain them at levels at which no more than a threshold level of ozone is produced per unit volume of input gas 202, monitors the flow rate of the input and/or output gas 202, 206 to provide a desired charge density, or dosage rate, at the target 132, monitors the humidity of the input and/or output gas 202, 206 to maintain the humidity within selected ranges, monitors the sensed altitude and/or barometric pressure to determine the molecular oxygen content of the input gas 202 and adjusts the flow rate of the input gas to provide a desired charge density at the target 132, monitors the charge density, or dosage rate, at the target 132 to maintain the dosage rate within selected ranges, monitors the input and/or output gas temperature to maintain the temperature above or below specified thresholds, and monitors the particulate count in the input or output gas 202, 206 to control particulate removal, such as by diverting all or part of the gas through a particulate removal device (not shown), such as an electrostatic precipitator or filter, before or after charging of the gas.

The user interface 128 may be any suitable interface for receiving commands from and providing feedback to the user or operator. The interface may be, for example, a set of keys, mouse, touch screen, or stylus for receiving tactile user input and a video display and/or speaker for providing visual or audible feedback to the operator. The input received from the operator can include the particular medical condition to be medicated for selection of the appropriate treatment protocol while the output provided to the operator can include instructions for performing the selected treatment protocol.

The target 132 can be any zoological biological tissue, for example mammalian or reptilian. The biological tissue is typically human. Examples of tissue that may be treated by the system 100 includes skin, eyes, ear canals, respiratory tract, mouth linings and gums, the tongue, or any other body orifice lining.

To inhibit charge buildup, the target 132 may be grounded and/or insulated electrically and held at a fixed, nonzero potential with respect to the charging device 204. The electric potential between the target 132 and the charging device 204 establishes an incidental electric field that assists in directing ions or charged particles to the target 132. If charge continues to buildup on the target, it can eventually repel the airborne charged particles and retard any beneficial physiological response. Preferably, the excess charge building up on the target is drained away to maintain a net flux of ions between the charging device and the target. When charge builds up, the potential difference between the target and charging device diminishes or vanishes and stimulation of the target stops.

Rather than grounding the target, the control module 124 can actively maintain a substantially fixed or constant voltage difference between the charging device 204 and the target 132 by effectively making the target 132 a reference or floating ground. This technique is well suited for portable or wearable treatment devices. Ungrounded, charge will bleed to space from the target at a slow rate so the process can work, but at a lower minimum level.

The system 100 is preferably configured to allow for controlled charged particle dosage rates (or quantities of electrical energy or of charged species) and/or droplet dosage rates (or the quantity or volume of additive 112) to the target 132 as a function of time. As will be appreciated, the dosage rate depends upon the type of medical condition being medicated. Dosage is preferably controlled by the polarity and limiting the net electrical charge transferred to the target 132. Dosage control is typically accomplished by controlling the space charge in proximity to the target 132 and the rate at which the charge transfers to the target 132. This is done by adjusting electric potential difference between the target 132 and the charging device 204 and/or by regulating the flow velocity of the charged, output gas 206 when emitted from the output gas directing device 220. Preferably, the space charge in proximity to the target 132 is maintained in the range of from about 10⁻¹⁶ to about 10⁻⁹ coulombs/CC, the delivery rate to the target surface in the range of from about 10³ to about 10⁹ ions or charged particles/cm²/sec, a non-zero electric field gradient and more preferably in the range of from about 15 to about 23 V/mm, the electric potential difference between the target and charging device in the range of from about −7 KVDC to about −13 KVDC volts, the system 100 voltage in the range of from about −7 KVDC to about −20 KVDC KVDC to generate electrostatic charge, the system 100 electrical energy output is about 10 joules or less, and the incident power imparted by the system 100 to the target is no more than about 10 mW/cm² with continuous exposure.

### Electrotherapeutic Device Configurations

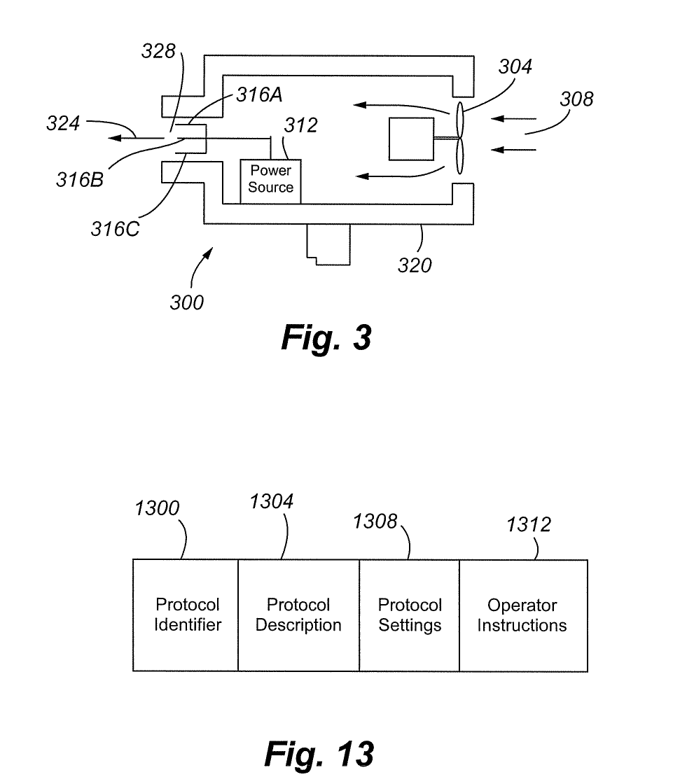

Referring now to FIG. 3, an electrotherapeutics treatment device 300 according to an embodiment is depicted. The device 300 includes a fan 304 to draw ambient air 308 into the device 300, a power source 312 electrically connected to a plurality of corona discharge needles 316a-c, which are preferably tungsten, to charge diatomic oxygen molecules in the air 308, and an outer housing 320 enclosing the foregoing components. The charged or ionized air 324 is directed though an outlet 328 of the housing towards a target (not shown) to be treated. This device is particularly adapted for home and office use. Rather than produce ions or charged liquid droplets that are conveyed to a specific target, this device 300 prepares a conditioned environment surrounding a cell, tissue, organism, or other target where exposure occurs by virtue of the presence of the target in the environment. This device 300 illustrates an aspect of the invention, which is the production of electrically charged species (atoms, droplets, particles, molecules, etc.) that are free and may be conveyed in a controlled fashion to the target biological tissue without the use of an electrode in intimate contact with the target tissue.

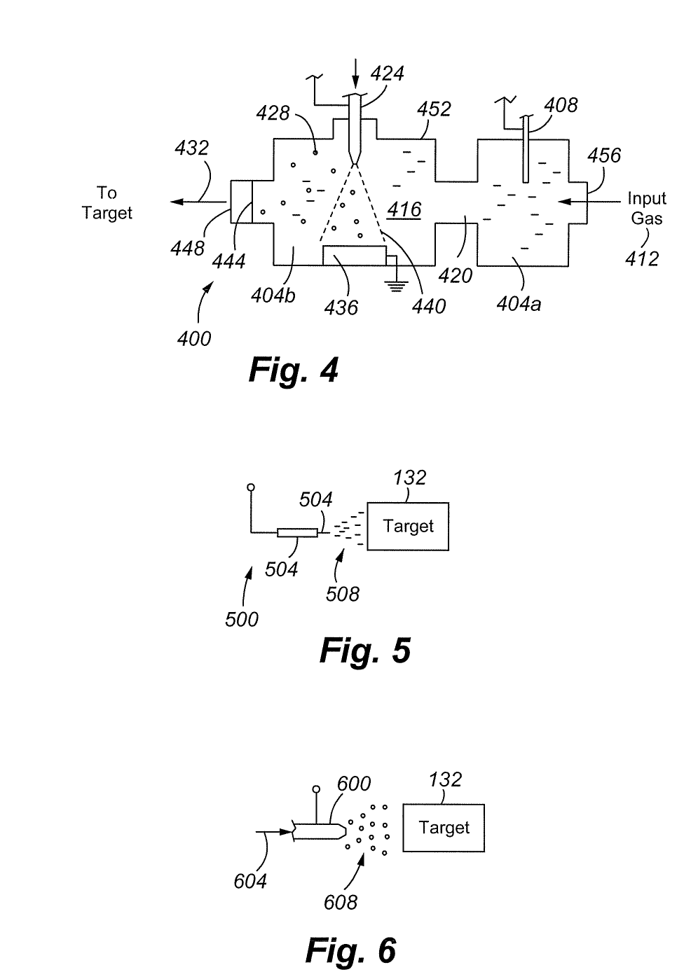

Referring now to FIG. 4, an electrotherapeutic treatment device 400 according to another embodiment is depicted. The device 400 includes first and second chambers 404a,b in fluid communication with one another via intervening passage 420. The first chamber 404a includes a corona discharge needle 408 to ionize an input gas 412 with ions (shown by the negative charge signs), which is preferably ambient air. The ionized or charged gas 416 is transported through the intervening passage 420 into the second chamber 404b. The second chamber 404b includes a nebulizer 424 to release charged droplets 428 into the ionized gas 416 and form an ionized and nebulized gas 432. Alternatively, the nebulizer 424 may be uncharged to provide a liquid pharmacological agent to the target 132. A grounded conductive or semiconductive plate 436 causes the charge to form a plume 440 in the gas 416 to effect distribution of the charged droplets in the gas 416. To remove solid particulates from the output gas, a filter 444 may be positioned in the outlet 448 defined by outer housing 452. The filter 444 can accumulate charge and deflect charged ions and droplets away from the outlet 448. The same can be said for any other cover over the outlet 448, such as a grating, grill, adjustable flow directing blades, or baffle. To avoid this, the filter may be removed entirely from the outlet 448 and repositioned in the inlet 456 or grounded to dissipate charge, and the outlet 448 can be made free of any grating, grill, flow directing blades, baffle, or other type of obstruction.

Referring now to FIG. 5, an electrotherapeutic treatment device 500 according to another embodiment is depicted. The device 500 includes a corona discharge needle 504 protruding from an insulated sheath 504. A cloud or plume 508 of ions is directed towards the target. Preferably, the time-of-flight to the target is preferably no more than about 2.5 minutes and even more preferably no more than about one minute. Charged particles are believed to have a persistence life of no more than about 30 seconds in free air before they combine with oppositely charged particles are neutralized. The persistence life can be impacted adversely by the humidity of the free air and the proximity of grounded surfaces.

Referring now to FIG. 6, an electrically charged nebulizer tip 600 is depicted. A saline additive 604 passes through the tip 600 to form a cloud or plume 608 of charged droplets. The droplets are directed towards the target. While not wishing to be bound by any theory, it is believed that the additive, under pressure, is transported into the tip 600 and atomized into droplets. As the droplets depart from the charged tip 600, they pick up electrons to provide the droplets with a negative charge.

### Electrotherapeutic Treatment Modalities

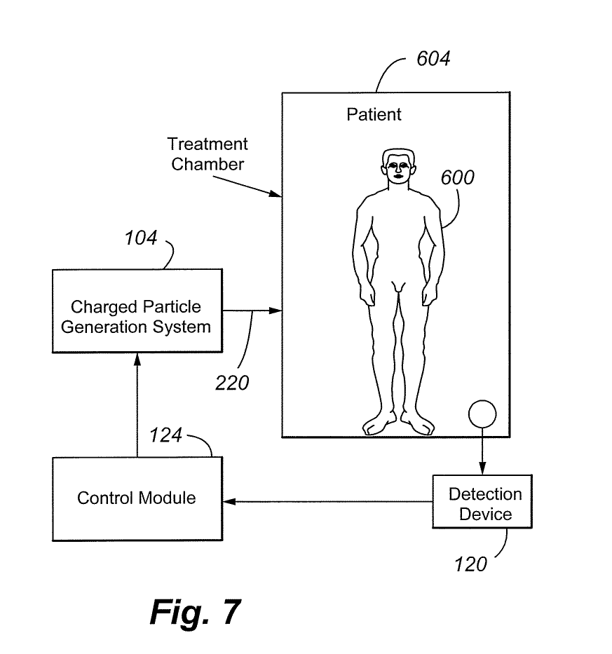

FIG. 7 depicts an embodiment of the electrotherapeutic treatment system that is particularly useful for treating a number of medical conditions, including burns, abrasions, respiratory ailments, cuts, and surgical wounds. The target, or patient 700, is contained wholly within containment in the form of a sealed treatment chamber 604. The charged particle generation system 104 generates a charged, output gas that is transported by the charged particle delivery system 108 and introduced to the treatment chamber 604. The charged particle generation system 104 could be in any form including those of FIGS. 3-6. A set of detection devices 120 monitor various parameters, discussed above, in the chamber 604 and provide signal feedback to the control module 124. The control module 124, based on the signal feedback, controls the operations of the charged particle generation system 104 to provide a selected dosage level to the treatment chamber 604.

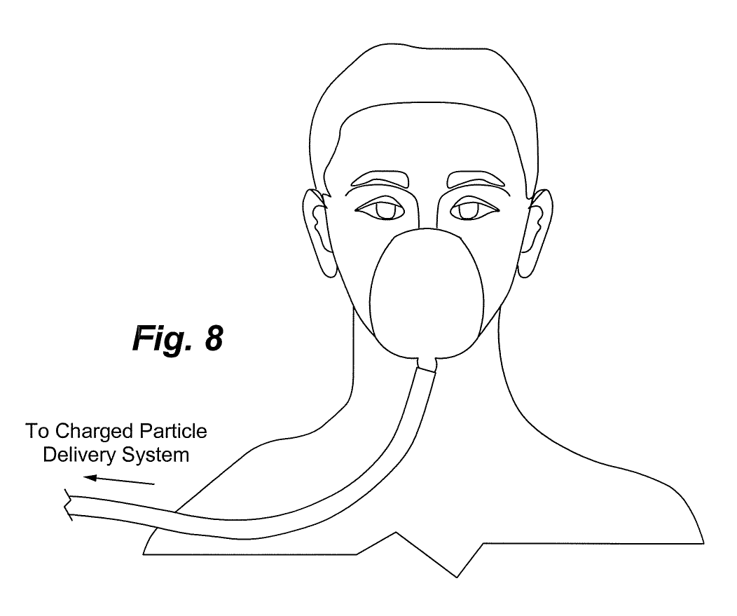

FIG. 8 depicts an embodiment of the electrotherapeutic treatment system that is particularly useful for treating respiratory ailments. The charged particle delivery system 108 includes a conduit leading to the charged particle generation system 104 and a mask 800 positioned over the patient's nasal and oral airways. The charged particle generation system 104 could be in any form including those of FIGS. 3-6.

Examples of biological respiratory tissue responses predicted as a result of receiving a dose of electrical energy delivered using gaseous airborne ions (GAIs) or electroaerosol droplets include: (i) stimulation of cilia on certain cells lining the respiratory tract to increase their beat frequency and thereby hasten transport of contaminates away from those surfaces; (ii) stimulation of certain secretory glands to produce less viscous secretions that are more readily transported or serve to dilute more viscous secretions already present and lying dormant on the tissues in a way that bolsters these thicker secretions' transport and removal; (iii) modification of electrical charge distributions of mucosa and mucus secretions in the respiratory tract that improve the elimination of mucus from those tracts or modify the physical properties of the mucus making its elimination from those tracts easier for the body's structures to accomplish for health benefit, particularly by establishing an electro osmotic gradient that helps move water from the lining tissues into thick viscous secretions contacting those tissues; and (iv) the potential destruction of pathogens topically attacking target tissues such as fungi, viruses, and bacteria to prevent or lessen infection or detrimental effects.

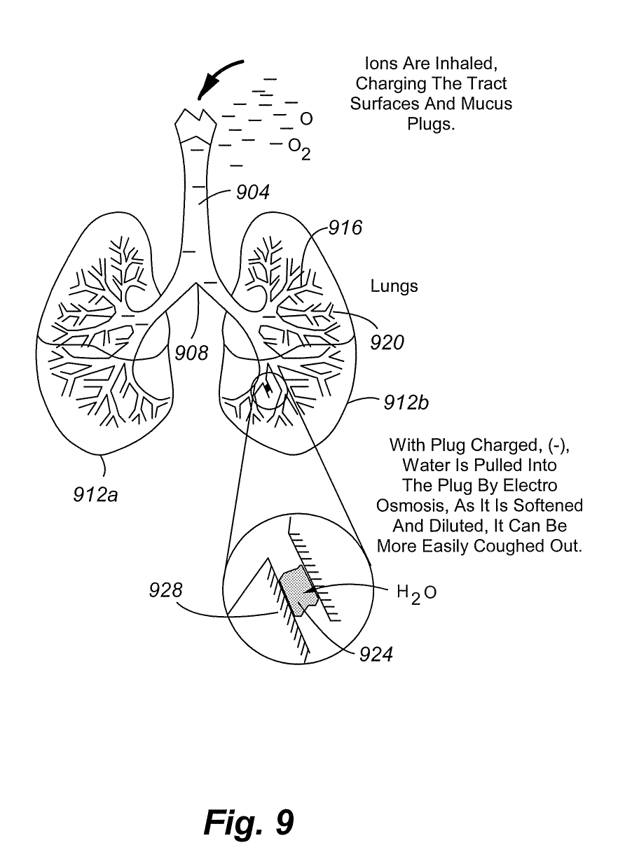

While not wishing to be bound by any theory, it is believed that the charged particles assist mucus plug removal in the manner shown in FIG. 9. FIG. 9 depicts a pair of lungs 900a,b. As will be appreciated, when one breathes air in through his or her nose or mouth, the air goes past the epiglottis and into the trachea 904. It continues down the trachea through his or her vocal cords in the larynx until it reaches the bronchi 908. From the bronchi, air passes into each lung 912a,b. The air then follows narrower and narrower bronchioles 916 until it reaches the alveoli 920. Within each air sac in the alveoli 920, the oxygen concentration is high, so oxygen passes or diffuses across the alveolar membrane (not shown) into the pulmonary capillary (not shown). As can be seen in the exploded view of a bronchi 908, airflow can be blocked by a mucus plug 924. In certain respiratory ailments, such as asthma and cystic fibrosis, mucus plugs can be difficult for a person to remove on his or her own. Cystic fibrosis, for example, is a genetic disorder in which mucus secreted by tissues lining the respiratory tract is abnormally thick and viscous, and is not easily removed by coughing or cilia transport mechanisms. As the lungs become increasingly obstructed, it becomes increasingly difficult for the cystic fibrosis sufferer to breathe. Once inhaled, ionized air or electroaerosol droplets travel through the respiratory tract and deposit their electrical charge on the tract linings, including onto the mucus plugs 924 themselves. Electrical charges deposited to the tissues directly will be bled away and will induce other beneficial responses such as increased cilia beat frequency, which will assist moving loosened mucus material out of the tract. Charge deposited on the plugs 924 will create a negative charge on the surface of the plug, in turn establishing a voltage gradient with respect to the tissue 928 contacting the plug 924. Assuming the plug 924 is negatively charged, water molecules will be induced by electro osmotic action to flow from the interstitial cellular matrix of the tissues 928 lining the respiratory tract into the negatively charged mucus plug to rehydrate the surface of the plug 924, thereby diluting its surface and causing it to soften and become less viscous. As the plug 924 softens and becomes less viscous, it becomes more easily transported through coughing and cilia action. Moreover, as the plug surface is diluted, a space of lower-viscosity mucus will surround the plug (the periciliary layer), allowing nearby cilia to more readily beat, in turn helping to clear the plug 924.

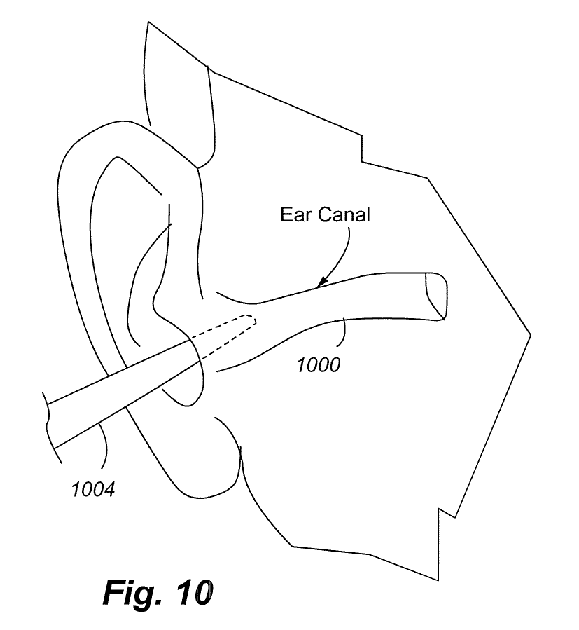

FIG. 10 shows an embodiment of the electrotherapeutic treatment system that is particularly useful for treating infections in the ear canal 1000. The charged particle delivery system 108 includes a conduit leading to the charged particle generation system 104 and an ear canal insert 1004 positioned in the ear canal 1000 to irrigate the ear canal with charged particles. The charged particle generation system 104 could be in any form including those of FIGS. 3-6.

FIG. 11 shows embodiment of the electrotherapeutic treatment system that is particularly useful for treating ocular ailments, such as dry eye syndrome and ocular infections and discomfort from wearing contact lenses. The charged particle generation and delivery systems are mounted on the frame 1100 of the glasses. The charged particle generation system 108 is positioned near an ear of the wearer with a charging device being positioned near each eye or only one eye. The charging device configuration is that set forth in FIG. 5.

The efficacy of the system of FIG. 11 can be magnified by administering electroactive eye drops or a crème including electrically conductive particles, such as carbon nanotubes. Carbon tubes are carbon lattices having a typical size of 1 to 4 nm in diameter. Owing to the small size, large electrical stresses can be generated by nanotubes. Consequently, charged particles can be emitted at low voltages, like those supplied by a battery or solar cell. The eye drops or crème including nanotubes are applied to the eye in a carefully controlled amount during or before charged particles are generated in spatial proximity to the eye. The electrical energy/charge transported to the eye by the charged particles can be conveyed to the nanotubes in close proximity to the ocular tissue.

### Selection of Appropriate Treatment Protocol

The control module 124 is preferably configured to permit the operator to treat a broad variety of treatment conditions using a single electrotherapeutic treatment system 100. This is done by the controller 124 using a lookup table to select an appropriate treatment protocol based on input by the operation through user interface 128.

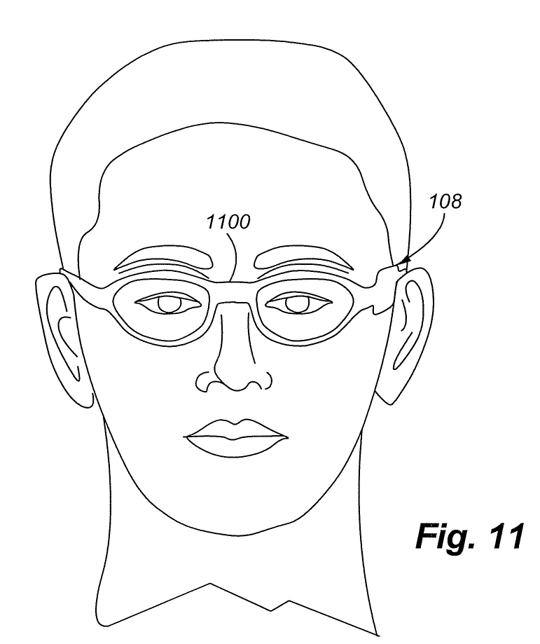

FIG. 13 depicts a set of data structures comprising an embodiment of the lookup table. The table includes a protocol identifier 1300 that identifies uniquely a particular protocol and, for each protocol identifier 1300, a corresponding protocol description 1304, set of protocol settings 1308, and operator instructions 1312. The protocol description 1304 can be an identifier, for example, of the particular medical conditions treated by the protocol. By way of illustration, the protocol description can be “dry eye syndrome” or “asthma”. The protocol settings 1308 refer to the settings of parameters used for the protocol. By way of illustration, a first protocol may have a first set of settings and a second protocol a different, second set of settings. The settings can be for parameters including exposure time of patient, the space charge in proximity to the patient, the ion or aerosol droplet delivery rate to the patient, the electric field gradient, the electric potential difference between the patient and charging device, the system voltage used to generate electrostatic charge, the system electrical energy output, the incident power imparted by the system to the patient over a selected time period, the voltage potential applied to and/or by the charging device, the electrical current flowing to the charging device, the flow rate of the input or output gases and/or of the additive, the humidity level of the input and/or output gases, whether particulate removal is to be used, a type or composition of additive to be used in aerosol droplets, and an amount of additive to be administered to the patient during the protocol. The operator instructions 1312 are the instructions provided to the operator before or during performance of the protocol. The instructions include, for instance, what attachments are to be used, manual settings, exposure times, and the like.

The operator may select a protocol using the interface 128 in many ways. The operator may select the protocol using a drop down menu, for instance. The operator may speak the condition to be treated and voice recognition software would then provide the text equivalent to the module 124 for protocol selection. The operator may type in the condition to be treated.

FIG. 12 depicts a process embodiment for selecting and administering a protocol.

In step 1200, the treatment protocol identifier is determined using input from the operator.

In step 1204, the treatment protocol identifier is mapped against the lookup table to determine the corresponding descriptive information for the protocol identifier.

In step 1208, the control module 124 configures the electrotherapeutic treatment system 100 as defined by the protocol settings 1308.

In step 1212, the control module 124 provides, via user interface 128, the instructions 1312 to the operator.

### Experimental

A patient suffering from Steven Johnson Syndrome (SJS) had severely damaged tissues, resulting in frequent and severe sinus infections, decreased tear film production or dry eye syndrome, dry mouth and asthma. The device of FIG. 3 was constructed, positioned within a few feet of the head of her bed, and placed into service at night while she rested. After two to three weeks, she felt noticeably better, experiencing increased tear film and saliva production, fewer asthma symptoms due to increased expulsion of mucus from her respiratory tract, and no further sinus infections. L ater examination by a physician indicated that she was experiencing tissue regeneration in her mucus membranes.

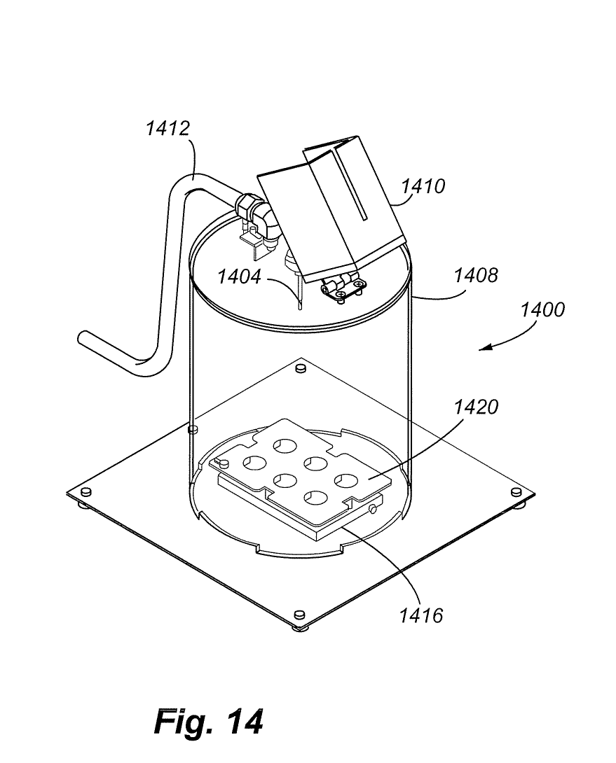

A test apparatus shown in FIG. 14 was constructed for experimentation with biological cells to validate whether predicted physiological responses would take place. The apparatus 1400 included a corona discharge needle 1404 positioned in a sealed enclosure 1408 and a shield 1410 protecting operators from being shocked during voltage application to the needle 1404. The needle 1404 ionizes diatomic oxygen molecules while air is introduced into the enclosure through conduit 1412. At the bottom of the enclosure 1408 is positioned an electrically insulating, six well sample tray 1416 with a grounded electrically conductive shield 1420 positioned between the tray 1416 and needle 1404 to prevent charge buildup on the tray and samples positioned in the wells. The device 1400 is connected to a Bertran™ 210-N10 high voltage supply. Two Keithley Instruments™ Model 485 picoammeters measured electrical charge. The corona discharge needle is a 3-inch length of 0.008-inch diameter tungsten wire. For the electrospray portion of the experiment, two Genie™ μL/hr syringe pumps and a commercial electrospray nozzle (not shown) were used. Atmospheric air was supplied to the enclosure 1408 using a June-Air™ OF301 oil-free air compressor and washer/humidifier system fabricated from a standard laboratory flask containing degassed and deionized water. The setup was not equipped with active temperature, humidity, and gas composition controls. Incoming chamber gases were passed through the washer/humidifier flask and an air filter to achieve reasonable test conditions and sterility in the enclosure.

For the corona discharge configuration, it was found that electric field strengths exceeding 33 V/mm resulted in the production of unwanted amounts of ozone in the enclosure. Measurements with the corona potential reduced to −7 KVDC showed a total ion flux, however, of 8.04×10⁹ ions/cm²/sec with an incidental electric field gradient of 23 V/mm. This ion flux exceeded that specified without the generation of appreciable amounts of ozone.

For the electrospray configuration, attempts to stabilize aerosol formation and eliminate ozone production while using compressed air as the nozzle propellant gas proved futile, so compressed high-purity nitrogen gas was used instead. Nitrogen gas flow was adjusted to achieve a stable trumpet-shaped plume, the hallmark of electrically active aerosols. Air was fed to the enclosure separately at 10 L/min to maintain the cell cultures, and liquid charge carrier (0.9% NaCl) was injected at the rate of 20 μL/min through the nozzle along with the nitrogen propellant gas.

With the test apparatus, a series of tests were conducted using bovine ocular cells, whole bovine eye organs (i.e., bulbs), immortalized SV-40 rabbit cornea epithelial cells (RCEC), and immortalized SV-40 human cornea epithelial cells (HCEC). Three physiological responses were investigated: 1) total mucin or glycoprotein secretion, 2) cell proliferation, and 3) cell migration.

### Mucin Secretion

Mucin secretion was tested by exposing entire bovine eyes to either airborne ions or electroaerosols and then assessing cell response following a period of time (cell responses require a period of time following exposure to a stimulus to fully transpire; they do not happen instantaneously).

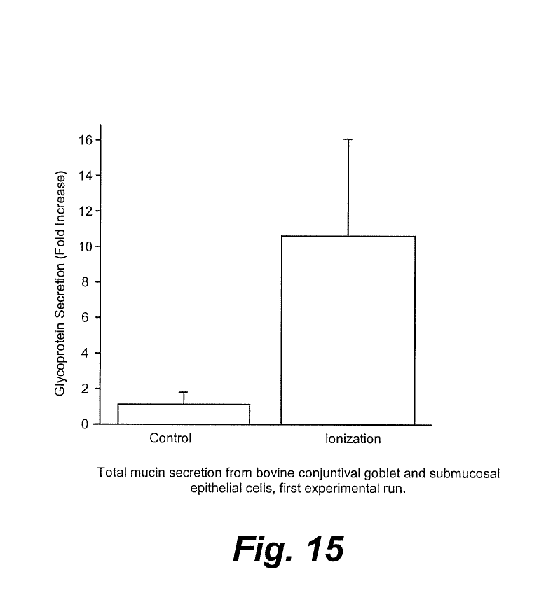

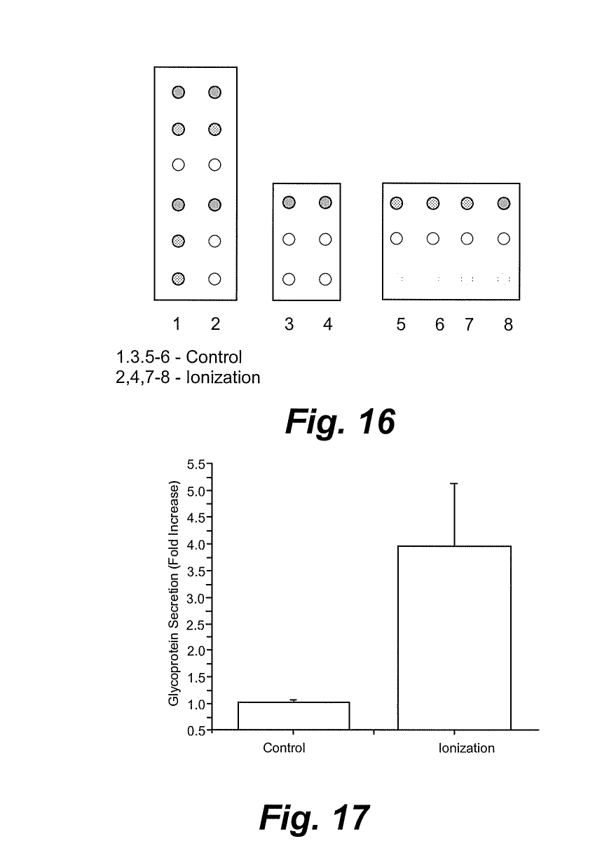

Available data are shown in FIGS. 15-17. This data shows that total mucin secretion from bovine conjunctival goblet and submucosal epithelial cells is increased by a factor of at least 6 upon exposure to negatively charged airborne gaseous ions (predominately negatively charged oxygen) for a period of 20 minutes.

Cell Proliferation

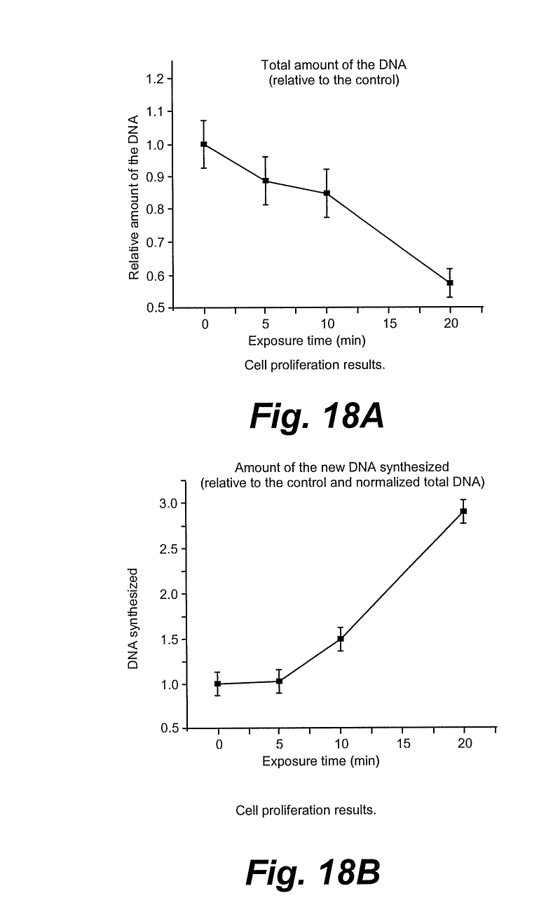

Data obtained for cell proliferation using the ³H-thymidine cell proliferation protocol is shown in FIGS. 18A and 18B. HCEC cells were exposed for 20 minutes to negatively charged gaseous airborne ions produced using corona discharge. FIGS. 18A and 18B show that total DNA present in the sample cell cultures decreased meaning that cells were detached and lost from the supporting membrane during the test so total cell count in the culture went down. For cells remaining on the culture membrane, their DNA was highly stimulated and found to be reproducing at a factor of 3 over baseline control. Data has not yet been collected for electroaerosol exposure.

Cells are held to substrates using predominantly positive charge. It is believed that introduction of negative charge to the culture causes weakened or damaged cells to detach by interfering with their ability to hold fast to the substrate. As an area opens up and cells are cleared away, surrounding cells, those that are securely attached, are stimulated through an unknown mechanism to begin proliferating to again cover the open area. This is the same phenomenon observed in the laboratory where cells proliferate until they reach confluence, or total coverage of an area somewhat akin to a tiled floor. This observed phenomena demonstrates that wound healing is plausible, as cell proliferation is stimulated strongly and is an essential function for would healing. It thus appears that the airborne transfer of electrical energy and/or charged-particles can stimulate this response through exposure to negatively charged gaseous air ions or electroaerosols.

Cell Migration

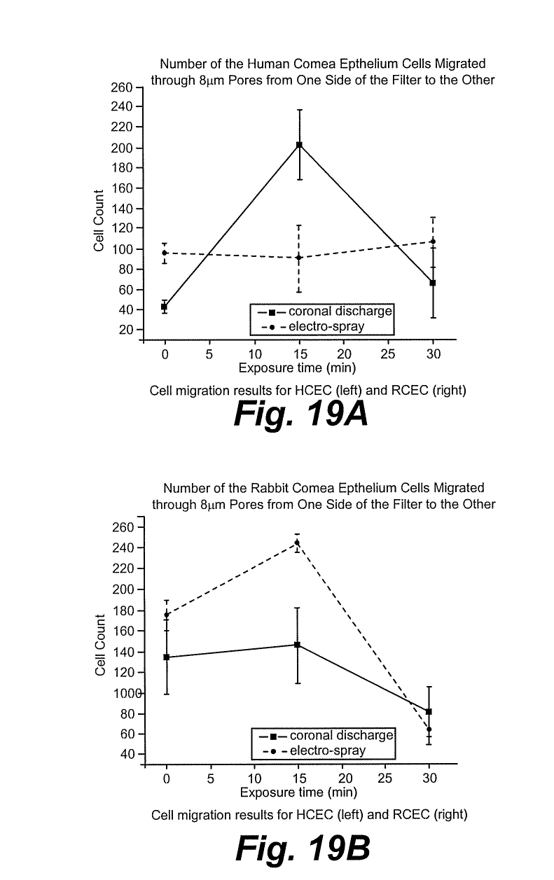

Data for cell migration was obtained by exposing RCECs and HCECs seeded to trans-well porous membranes to look for migration. Well membranes have 8 μm diameter pores; stimulated and actively migrating cells squeeze through these openings to the underside of the membrane opposite the side exposed to the stimulus. Exposure times and results are shown in FIGS. 19A and 19B. Results were obtained using before and after cell counts obtained with a 10× objective microscope and staining techniques.

These results indicate that human cells are more responsive to gaseous air ions generated through corona discharge than electroaerosols, and they indicate that there may be an optimal exposure time for maximized beneficial effect of approximately 15 minutes or so. This observed phenomena demonstrates that wound healing is plausible, as cell migration is stimulated strongly and is an essential function for would healing. Thus, the treatment system 100 can stimulate this response through exposure to negatively charged gaseous air ions or electroaerosols.

In the above tests, ionization using a corona discharge needle appeared to have much greater efficacy than electroaerosols. Compared to the positive results realized using ionization, electroaerosol test results were generally inconclusive.

Additional Testing

An additional set of tests was conducted to confirm a prediction that a mobile, i.e. floating, lipid layer could be forced to expand and cover an increased area by charging it electrically. A layer of 0.9% NaCl (physiological saline) solution was placed into a grounded conductive pan, and a small droplet of oil floated on the surface. Gaseous air ions were then directed at the oil droplet so that it became electrically charged. Because lipids are generally electrical insulators, charges reaching the drop became entrained. Charges reaching the highly conductive saline were, in contrast, immediately conducted to ground and removed. Because like charges repel and charges accumulating on the oil droplet can not be conducted away (oils are insulators), the drop expands until surface tension effects prevail and expansion halts. For the light oil used, the effective expansion was 12-fold.

While not wishing to be bound by any theory, it is believed that this phenomenon may potentially be used to limit evaporative effects from the human ocular tear film by spreading the lipid layer portion of the tear film, countering surface tension effects to delay breakup. The discovery suggests a way to help combat tear film disorders in the human eye for improved ocular comfort and health.

A member of variations and modifications of the invention can be used. It would be possible to provide for some features of the invention without providing others.

For example in one alternative embodiment, a conductive material is used on or below the surface of the target tissue to enhance the electrotherapeutic treatment effect of contact with charged particles. The conductive gel, crème, or coating is able to spread the electrical energy over a wider area and also can carry medicinal compounds to enhance treatment efficacy.

In another alternative embodiment, the conductive gel or aerosol droplets emitted from the charging device include bioactive compounds that are activated or deactivated with the presence of electrical energy or cations or anions, whose uptake into the tissues is enhanced, or whose rate of uptake into the tissues is controlled precisely. It is well documented that biological functions are triggered by the movement and release of ions within and between cells and tissues.

In yet another alternative embodiment, corona discharge or electrospray is used to neutralize electrical effects within a gas stream administering atomized droplets of a pharmaceutical agent. It is known to use nebulized sprays as a modality of administering liquid pharmaceutical preparations. Applying an electrical charge to the droplets can prevent undesired agglomeration of the pharmaceutical drops or species in a cloud to ensure that pharmaceutical agents are delivered without interference and/or enhance a function of the nebulized pharmaceutical agents upon the tissues of humans or animals. It is possible to actively control space charge or eliminate it altogether if necessary should its presence cause an undesirable effect.

In yet another alternative embodiment, polarity of an applied charge and/or the ratio of negative and positive charge is changed in a time-dependent fashion. For example, during a first time interval a first ratio of negative-to-positive charge is used and in a second, later time interval a second, different ratio of negative-to-positive charge is used. Alternatively, during the first time interval a first dosage level of charge is used, and in the second time interval a second, different dosage level of the same charge is used. Changing polarity or dosage rate of an applied charge can impact how certain tissues are affected by electrical energy/charge transfer.

In yet another alternative embodiment, the beneficial effects of ion or electroaerosol exposure can be augmented by infusing, injecting, or absorbing an electroactive component into the tissues themselves. This might, for instance, include the use of nanotubes, nanoparticles, or metal particulates, such as silver, to enhance electrical effects.

The present invention, in various embodiments, includes components, methods, processes, systems and/or apparatus substantially as depicted and described herein, including various embodiments, subcombinations, and subsets thereof. Those of skill in the art will understand how to make and use the present invention after understanding the present disclosure. The present invention, in various embodiments, includes providing devices and processes in the absence of items not depicted and/or described herein or in various embodiments hereof, including in the absence of such items as may have been used in previous devices or processes, e.g., for improving performance, achieving ease and\or reducing cost of implementation.

The foregoing discussion of the invention has been presented for purposes of illustration and description. The foregoing is not intended to limit the invention to the form or forms disclosed herein. In the foregoing Detailed Description for example, various features of the invention are grouped together in one or more embodiments for the purpose of streamlining the disclosure. The features of the embodiments of the invention may be combined in alternate embodiments other than those discussed above. This method of disclosure is not to be interpreted as reflecting an intention that the claimed invention requires more features than are expressly recited in each claim. Rather, as the following claims reflect, inventive aspects lie in less than all features of a single foregoing disclosed embodiment. Thus, the following claims are hereby incorporated into this Detailed Description, with each claim standing on its own as a separate preferred embodiment of the invention.

Moreover, though the description of the invention has included description of one or more embodiments and certain variations and modifications, other variations, combinations, and modifications are within the scope of the invention, e.g., as may be within the skill and knowledge of those in the art, after understanding the present disclosure. It is intended to obtain rights which include alternative embodiments to the extent permitted, including alternate, interchangeable and/or equivalent structures, functions, ranges or steps to those claimed, whether or not such alternate, interchangeable and/or equivalent structures, functions, ranges or steps are disclosed herein, and without intending to publicly dedicate any patentable subject matter.

What is claimed is:

1. A treatment method, comprising:
(a) electrically charging at least one of a plurality of (i) electrically charged diatomic oxygen and/or water molecules and (ii) electrically charged droplets in an input gas stream to form a charged gas stream, while controlling ozone production; and
(b) providing the charged gas stream to a living organism to be treated.

2. The method of claim 1, wherein the input gas stream comprises particulates, wherein, in step (b), the charged gas stream is not passed through an obstruction in a path of flow of the charged gas stream, wherein the particulates are not removed before the providing step (b), and wherein, in the charging step (a), an electrostatic voltage of a charging device producing the at least one of a plurality of (i) electrically charged diatomic oxygen and/or water molecules and (ii) electrically charged droplets is maintained at a substantially constant voltage and/or frequency.

3. The method of claim 1, wherein, in the providing step (b), the charged gas stream is provided to a substantially sealed area comprising at least a part of the living organism to be treated, wherein at least one of the living organism and containment defining the substantially sealed area is grounded, and wherein the electrically charging step is performed at a distance from the living organism without a nongaseous conductive path being positioned between a charging device performing step (a) and the living organism.

4. The method of claim 1, wherein the electrically charging step (a) is performed by a corona discharge electrode, wherein the at least one of a plurality of (i) electrically charged diatomic oxygen and/or water molecules and (ii) electrically charged droplets is electrically charged diatomic oxygen and/or water molecules, wherein the electrode is maintained at a substantially constant voltage and/or frequency, and wherein a voltage applied to the electrode is no more than about 20 kvolts.

5. The method of claim 1, wherein the electrically charging step (a) is performed by a nebulizer nozzle, wherein the at least one of a plurality of (i) electrically charged diatomic oxygen and/or water molecules and (ii) electrically charged droplets is electrically charged droplets, wherein the nozzle is maintained at a substantially constant voltage and/or frequency, and wherein a voltage applied to the nozzle is no more than about 20 kvolts.

6. The method of claim 1, further comprising:
(c) controlling a humidity level of at least one of the input and charged gas streams.

7. The method of claim 1, further comprising:
(c) controlling a dosage rate of the at least one of a plurality of (i) electrically charged diatomic oxygen and/or water molecules and (ii) electrically charged droplets provided to the living organism.

8. The method of claim 1, further comprising at least one of the following steps:
(c1) monitoring the voltage and/or current provided to a charging device performing step (a) and maintaining the voltage and/or current at a selected level sufficient to inhibit substantially ozone production,
(c2) monitoring the flow rate of the input and/or charged gas and maintaining a desired charge density, or dosage rate, at the living organism,
(c3) monitoring the sensed altitude and/or barometric pressure to determine a molecular oxygen content of the input gas and adjusting the flow rate of the input gas to provide a desired charge density at the living organism,
(c4) monitoring the charge density, or dosage rate, at the living organism and maintaining the dosage rate within a selected range,
(c5) monitoring the input and/or charged gas temperature and maintaining the temperature above or below specified thresholds,
(c6) monitoring a particulate count in the input and/or charged gas and diverting all or part of the gas through a particulate removal device before and/or after charging of the gas to control a particulate level,
(c7) maintaining a substantially constant voltage difference between the charging device and living organism by using the living organism as a reference ground.

9. The method of claim 1, further comprising:
(c) performing at least one of the following substeps:
(i) maintaining a space charge in proximity to the living organism in the range of from about 10⁻¹⁶ to about 10⁻⁹ coulombs/CC,
(ii) maintaining a delivery rate to the living organism in the range of from about 10³ to about 10⁹ ions or charged particles/cm²/sec,
(iii) maintaining a non-zero electric field gradient (iv) maintaining an electric field strength of no more than about 33 volts/mm, and
(v) maintaining a content of ozone in the charged gas stream to no more than about 80 ppb.

10. The method of claim 1, further comprising:
(c) determining a treatment protocol to be employed;
(d) based on the determined treatment protocol, determining a corresponding set of settings to be employed in steps (a) and/or (b); and
(e) configuring, before step (a), a treatment system in accordance with the corresponding set of settings.

11. A living organism treated by the method of claim 1.

12. A treatment method, comprising:
(a) electrically charging at least one of a atomic particles in an input gas stream to form a charged gas stream, while controlling ozone production; and
(b) providing the charged gas stream to a living organism to be treated.

13. The method of claim 12, wherein the input gas stream comprises particulates, wherein, in step (b), the charged gas stream is not passed through an obstruction in a path of flow of the charged gas stream, wherein the particulates are not removed before the providing step (b), and wherein, in the charging step (a), an electrostatic voltage of a charging device producing the electrically charged atomic particles is maintained at a substantially constant voltage and/or frequency.

14. The method of claim 12, wherein, in the providing step (b), the charged gas stream is provided to a substantially sealed area comprising at least a part of the living organism to be treated, wherein at least one of the living organism and containment defining the substantially sealed area is grounded, and wherein the electrically charging step is performed at a distance from the living organism without a nongaseous conductive path being positioned between a charging device performing step (a) and the living organism.

15. The method of claim 12, wherein the electrically charging step (a) is performed by a corona discharge electrode, wherein the charged atomic particles are at least one of a plurality of electrically charged diatomic oxygen and water molecules, wherein a charging device performing step (a) is maintained at a substantially constant voltage and/or frequency, and wherein a voltage applied to the electrode is no more than about 20 kvolts.

16. The method of claim 12, further comprising:
(c) controlling a humidity level of at least one of the input and charged gas streams.

17. The method of claim 12, further comprising:
(c) controlling a dosage rate of the electrically charged atomic particles provided to the living organism.

18. The method of claim 12, further comprising at least one of the following steps:
(c1) monitoring the voltage and/or current provided to a charging device performing step (a) and maintaining the voltage and/or current at a selected level sufficient to inhibit substantially ozone production,
(c2) monitoring the flow rate of the input and/or charged gas and maintaining a desired charge density, or dosage rate, at the living organism,
(c3) monitoring the sensed altitude and/or barometric pressure to determine a molecular oxygen content of the input gas and adjusting the flow rate of the input gas to provide a desired charge density at the living organism,
(c4) monitoring the charge density, or dosage rate, at the living organism and maintaining the dosage rate within a selected range,
(c5) monitoring the input and/or charged gas temperature and maintaining the temperature above or below specified thresholds,
(c6) monitoring a particulate count in the input and/or charged gas and diverting all or part of the gas through a particulate removal device before and/or after charging of the gas to control a particulate level,
(c7) maintaining a substantially constant voltage difference between the charging device and living organism by using the living organism as a reference ground.

19. The method of claim 12, further comprising:
(c) performing at least one of the following substeps:
(i) maintaining a space charge in proximity to the living organism in the range of from about 10⁻¹⁶ to about 10⁻⁹ coulombs/CC,
(ii) maintaining a delivery rate to the living organism in the range of from about 10³ to about 10⁹ ions or charged particles/cm²/sec,
(iii) maintaining a non-zero electric field gradient,
(iv) maintaining an electric field strength of no more than about 33 volts/mm, and
(v) maintaining a content of ozone in the charged gas stream to no more than about 80 ppb.

20. The method of claim 12, further comprising:
(c) determining a treatment protocol to be employed;
(d) based on the determined treatment protocol, determining a corresponding set of settings to be employed in steps (a) and/or (b); and
(e) configuring, before step (a), a treatment system in accordance with the corresponding set of settings.

21. An electrotherapeutic treatment device, comprising:
(a) a charging device operable to electrically charge at least one of a plurality of (i) electrically charged diatomic oxygen and/or water molecules and (ii) electrically charged droplets in an input gas stream to form a charged gas stream, while controlling ozone production; and
(b) an output gas directing device operable to provide the charged gas stream to a living organism to be treated.

22. The device of claim 21, wherein the input gas stream comprises particulates, wherein the charged gas stream is not passed through an obstruction in a path of flow of the charged gas stream, wherein the particulates are not removed before the charged gas stream is provided to the living organism, and wherein an electrostatic voltage of the charging device producing the at least one of a plurality of (i) electrically charged diatomic oxygen and/or water molecules and (ii) electrically charged droplets is maintained at a substantially constant voltage and/or frequency.

23. The device of claim 21, wherein the output gas directing device provides the charged gas stream to a substantially sealed area comprising at least a part of the living organism to be treated, wherein at least one of the living organism and containment defining the substantially sealed area is grounded, and wherein the charging device is positioned at a distance from the living organism without a nongaseous conductive path being positioned between the charging device and the living organism.

24. The device of claim 21, wherein the charging device is a corona discharge electrode, wherein the at least one of a plurality of (i) electrically charged diatomic oxygen and/or water molecules and (ii) electrically charged droplets is electrically charged diatomic oxygen and/or water molecules, wherein the electrode is maintained at a substantially constant voltage and/or frequency, and wherein a voltage applied to the electrode is no more than about 20 kvolts.

25. The device of claim 21, wherein the charging device is a nebulizer nozzle, wherein the at least one of a plurality of (i) electrically charged diatomic oxygen and/or water molecules and (ii) electrically charged droplets is electrically charged droplets, wherein the nozzle is maintained at a substantially constant voltage and/or frequency, and wherein a voltage applied to the nozzle is no more than about 20 kvolts.

26. The device of claim 21, further comprising:
(c) a humidity source operable to control a humidity level of at least one of the input and charged gas streams.

27. The device of claim 21, further comprising:
(c) a control module operable to control a dosage rate of the at least one of a plurality of (i) electrically charged diatomic oxygen and/or water molecules and (ii) electrically charged droplets provided to the living organism.

28. The device of claim 21, further comprising a control module operable to perform at least one of the following operations:
(i) monitoring the voltage and/or current provided to the charging device and maintaining the voltage and/or current at a selected level sufficient to inhibit substantially ozone production,
(ii) monitoring the flow rate of the input and/or charged gas and maintaining a desired charge density, or dosage rate, at the living organism,
(iii) monitoring the sensed altitude and/or barometric pressure to determine a molecular oxygen content of the input gas and adjusting the flow rate of the input gas to provide a desired charge density at the living organism,
(iv) monitoring the charge density, or dosage rate, at the living organism and maintaining the dosage rate within a selected range,
(v) monitoring the input and/or charged gas temperature and maintaining the temperature above or below specified thresholds,
(vi) monitoring a particulate count in the input and/or charged gas and diverting all or part of the gas through a particulate removal device before and/or after charging of the gas to control a particulate level,
(vii) maintaining a substantially constant voltage difference between the charging device and living organism by using the living organism as a reference ground.

29. The device of claim 21, further comprising a control module operable to perform at least one of the following operations:
(i) maintaining a space charge in proximity to the living organism in the range of from about 10⁻¹⁶ to about 10⁻⁹ coulombs/CC,
(ii) maintaining a delivery rate to the living organism in the range of from about 10³ to about 10⁹ ions or charged particles/cm²/sec,
(iii) maintaining a non-zero electric field gradient,
(iv) maintaining an electric field strength of no more than about 33 volts/mm, and
(v) maintaining a content of ozone in the charged gas stream to no more than about 80 ppb.

30. The device of claim 21, further comprising a control module operable to:
(i) determine a treatment protocol to be employed;
(ii) based on the determined treatment protocol, determine a corresponding set of settings to be employed in steps (a) and/or (b); and
(iii) configure, before step (a), a treatment system in accordance with the corresponding set of settings.
